(19) United States
(12) Patent Application Publication
(10) Pub. No.: US 2008/0234485 A1
(43) Pub. Date: Sep. 25, 2008

(54) 1-Benzyl-4-[ (5,6-Dimethoxy-1-Indanon) -2-Yl] Methylpiperidine Hydrobromide or Crystals Thereof

(76) Inventors: Kazuhide Ashizawa, Tsukuba (JP); Takashi Hasebe, Andover, MA (US); Akio Imai, Kamisu (JP)

Correspondence Address: BIRCH STEWART KOLASCH & BIRCH, PO BOX 747, FALLS CHURCH, VA, 22040-0747, US

(21) Appl. No.: 11/995,975

(22) PCT Filed: Feb. 1, 2007
(86) PCT No.: PCT/US2006/028035

§ 371 Date: Jan. 17, 2008

Related U.S. Application Data
(60) Provisional application No. 60/701,031, filed on Jul. 21, 2005.

(30) Foreign Application Priority Data
Jul. 20, 2005 (JP) 2005-210184

(51) Int. Cl. C07D 211/10

(52) U.S. Cl. 546/205

(57) ABSTRACT

1-Benzyl-4-[(5,6-dimethoxy-1-indanon)-2-yl]methylpiperidine hydrobromide or a solvate thereof.

### TECHNICAL FIELD

The present invention relates to donepezil hydrobromide, or a crystal thereof, which possesses an acetylcholinesterase inhibitory action and which is effective as a preventative drug or as a therapeutic drug for various types of senile dementia or the like.

### BACKGROUND ART

Donepezil hydrochloride (chemical name: 1-benzyl-4-[(5,6-dimethoxy-1-indanon)-2-yl]methylpiperidine hydrochloride is a therapeutic medicine for various kinds of senile dementia that has an acetylcholinesterase inhibitory action and that is extremely useful as a preventative agent or as a therapeutic agent in particular for Alzheimer's type senile dementia (Patent Document 1).

As inorganic salts of donepezil, 1-benzyl-4-[(5,6-dimethoxy-1-indanon)-2-yl]methylpiperidine hydrochloride and a crystal thereof (Patent Document 2) and 1-benzyl-4-[(5,6-dimethoxy-1-indanone)-2-yl]methylpiperidine hydrobromide and a crystal thereof (Patent Document 3) are known. Patent Document 3 discloses two kinds of crystal polymorphisms of 1-benzyl-4-[(5,6-dimethoxy-1-indanon)-2-yl]methylpiperidine hydrobromide.

The present invention includes two kinds of crystals of 1-benzyl-4-[(5,6-dimethoxy-1-indanon)-2-yl]methylpiperidine hydrobromide. Although the crystal obtained in Example 3 accords with Form II of the crystal disclosed in Patent Document 3, the crystal obtained in Example 2 is completely different from that disclosed in Patent Document 3 in crystal structure and hence a novel crystal.

[Patent Document 1] JP-A-64-79151

[Patent Document 2] WO 97/46527A

[Patent Document 3] WO2004/099142A

### DISCLOSURE OF INVENTION

Salts and crystals which are used as pharmaceutical raw materials need to be easily handled during industrial preparation.

As a result of intensive research, the present inventors completed at the present invention through their discovery of a novel salt below. That is, the present invention relates to donepezil hydrobromide, a solvate thereof or a crystal thereof.

Namely, the present invention includes the followings:

(1) 1-Benzyl-4-[(5,6-dimethoxy-1-indanon)-2-yl]methylpiperidine hydrobromide or a solvate thereof;
(2) A crystal of 1-benzyl-4-[(5,6-dimethoxy-1-indanon)-2-yl]methylpiperidine hydrobromide or a solvate thereof having diffraction peaks at diffraction angles (2θ±0.2°) of 6.6° and 26.9° in powder X-ray diffraction;
(3) The crystal described in above-mentioned (2), further having diffraction peaks at diffraction angles (2θ±0.2°) of 21.1° and 21.6° in powder X-ray diffraction;
(4) A crystal of 1-benzyl-4-[(5,6-dimethoxy-1-indanon)-2-yl]methylpiperidine hydrobromide or a solvate thereof having diffraction peaks at diffraction angles (2θ±0.2°) of 21.5° and 24.4° in powder X-ray diffraction;
(5) The crystal described in above-mentioned (4), further having diffraction peaks at diffraction angles (2θ±0.2°) of 10.2° and 18.2° in powder X-ray diffraction;
(6) A method for preparing the crystal described in above-mentioned (4), characterized by performing crystallization with one or two solvents selected from the group consisting of alcohols and water;
(7) A pharmaceutical comprising the salt, solvate thereof or crystal thereof described in any of the above-mentioned (1) to (5);
(8) A preventative or therapeutic agent for a disorder to which an acetylcholinesterase inhibitory action is effective, comprising as an active ingredient the salt, solvate thereof or crystal thereof described in any of the above-mentioned (1) to (5);
(9) A preventative or therapeutic agent for senile dementia, comprising as an active ingredient the salt, solvate thereof or crystal thereof described in any of the above-mentioned (1) to (5);
(10) A preventative or therapeutic agent for Alzheimer's disease, comprising as an active ingredient the salt, solvate thereof or crystal thereof described in any of the above-mentioned (1) to (5); and
(11) A pharmaceutical composition comprising the salt, solvate thereof or crystal thereof described in any of the above-mentioned (1) to (5).

### BRIEF DESCRIPTION OF DRAWINGS

FIG. 1 is a diagram illustrating the powder X-ray diffraction pattern of the crystals obtained in Example 2.

FIG. 2 is a diagram illustrating the powder X-ray diffraction pattern of the crystals obtained in Example 3.

### BEST MODE FOR CARRYING OUT THE INVENTION

Hereafter, the meaning of the terms and reference characters used herein will be described and the present invention will then be described in detail.

“Donepezil” means 1-benzyl-4-[(5,6-dimethoxy-1-indanone)-2-yl]methylpiperidine and crystal polymorphs may be present for the crystals of the salt or a solvate thereof according to the present invention. However, the present invention is not limited, and the crystal of the present invention may be a single crystal form or a mixture thereof.

Since an error in the range of ±0.2° may occur in diffraction angles (2θ)±0.2° in powder X-ray diffraction, in general, the value of the above diffraction angle should be understood as including values in a range of around ±0.2°. Therefore, the present invention includes not only crystals whose diffraction angles of the peaks in powder X-ray diffraction perfectly match, but also crystals whose diffraction angles of the peaks match within an error of around ±0.2°.

“Alcohols” means a C₁₋₆ alkyl alcohols. Specific examples include methanol, ethanol, isopropanol, n-propanol and the like.

“Ethers” means a di C₁₋₆ alkyl ethers or a cyclic ether. Specific examples include dimethyl ether, diisopropyl ether, t-butyl methyl ether, tetrahydrofuran and the like.

In “1-benzyl-4-[(5,6-dimethoxy-1-indanon)-2-yl]methylpiperidine hydrobromide or a solvate thereof”, the solvent is not especially limited as long as it is formed by a salt of the present invention and the solvent, it is a form in which the solvent forms a solvate at an appropriate ratio between 0.1 and 5 molecules per 1 molecule of the compound. The solvent for the solvate is not especially limited, and examples include solvents employed in the preparation of the salt of the present invention and the crystal thereof (alcohols, ethers, or the like) and water or the like. Preferable examples include from one to three solvents (if combining plural solvents, a mixture at any arbitrary ratio) selected from the group consisting of water, diisopropyl ether and ethanol. More preferable examples include water and the like.

For the salt according to the present invention, the ratio between donepezil and hydrogen bromide is not especially limited. The hydrogen bromide forms the salt at a ratio between 0.5 to 2 molecules (preferably about 1 molecule per 1 molecule of the subject compound) per 1 molecule of donepezil.

The salt, solvate thereof, or crystal thereof, according to the present invention can be prepared according the methods described below. However, the method for preparing the salt, solvate thereof or crystal thereof, according to the present invention is not limited to these methods.

### Preparation Process

### [Preparation of Donepezil Hydrobromide]

In the preparation method according to the present invention, donepezil (1-benzyl-4-[(5,6-dimethoxy-1-indanone)-2-yl]methylpiperidine) used as a starting material can be prepared according to the methods (or similar) described in a patent document (WO 99/29668A).

### (Operation 1)

Donepezil, a solvent and hydrogen bromide are mixed together and dissolved at room temperature or under heating. In this dissolution process, the order to add donepezil, the solvent and the acid together is not particularly limited, and this process may be performed with stirring or standing.

### (Operation 2)

After this mixture dissolves, donepezil hydrobromide can be obtained according to the below method and the like.

(1) Distilling off the solvent from the mixture under atmospheric pressure or reduced pressure.
(2) Stirring or standing the mixture at the dissolution temperature.
(3) Cooling the mixture from the dissolution temperature, and then stirring or standing.
(4) Adding an anti-solvent to the mixture at the dissolution temperature, and then stirring or standing.
(5) Adding an anti-solvent to the mixture, cooling, and then stirring or standing.

### (Operation 3)

When donepezil hydrobromide is obtained as a precipitate, a solid or the like, the precipitate, etc., can be washed with a suitable solvent. In addition, the obtained precipitate or residue can also be dried, as necessary, under atmospheric pressure or reduced pressure at room temperature or under heating.

The length of time of the process for obtaining donepezil hydrobromide (the above Operation 2) is not particularly limited, but it is preferably 1 hour to 3 days, and more preferably 1 to 24 hours. In addition, the cooling temperature or cooling rate for obtaining donepezil hydrobromide is not particularly limited.

### [Preparation of Crystal of Donepezil Hydrobromide]

Donepezil hydrobromide can also be obtained as crystals by carrying out the above (Operation 1) to (Operation 3).

In addition, donepezil hydrobromide can also be obtained as crystals by mixing and dissolving donepezil hydrobromide with a solvent, and then (1) distilling off the solvent from the mixture under atmospheric pressure or reduced pressure; (2) stirring or standing the mixture at the dissolution temperature; (3) cooling the mixture from the dissolution temperature, and then stirring or standing; (4) charging the mixture with an anti-solvent at the dissolution temperature, and then stirring or standing; or (5) charging the mixture with an anti-solvent, cooling, and then stirring or standing.

Hydrogen bromide may be either gaseous or in solution, although a solution of hydrogen bromide is preferred, and an aqueous solution of hydrogen bromide is more preferred.

The above solvent is not particularly limited. Specific examples include one or plural solvents selected from the group consisting of water, alcohols (e.g., methanol, ethanol, isopropyl alcohol and the like), esters (e.g., methyl acetate, ethyl acetate and the like), ketones (e.g., acetone and the like), nitrites (e.g., acetonitrile and the like), benzene, toluene, cyclic ethers (e.g., dioxane, tetrahydrofuran and the like), N,N-dimethylformamide, dimethylsulfoxide, and halocarbons (e.g., methylene chloride and the like). If using plural solvents, such solvents may be added as a mixed solvent, or each solvent may be added individually.

The heating temperature upon dissolving the mixture of donepezil, hydrogen bromide and a solvent is not particularly limited, but is preferably from 20 to 80° C.

The temperature upon cooling after dissolving the mixture consisting of donepezil, hydrogen bromide and a solvent is not particularly limited, but is preferably from −20 to 40° C.

The amount of solvent used is not particularly limited, but is preferably suitably selected between a lower limit set at the amount where donepezil dissolves by heating and an upper limit set at the amount where the crystal yield does not remarkably reduce. More preferable is from 4 to 30 times the amount (v/w) by volume ratio with respect to the weight of donepezil.

The amount of hydrogen bromide used is not particularly limited as long as the amount is equal to or greater than the amount of donepezil. Preferable is from 1 to 3 times the amount of donepezil in terms of molar ratio, and more preferable is from about 1 to 1.5 times the amount of donepezil in terms of molar ratio.

When donepezil hydrobromide is obtained as crystals, a seed crystal (a crystal of the desired donepezil hydrobromide) can also be added prior to crystal precipitation. The temperature at which the seed crystal is added is not particularly limited, but is preferably 60° C. or less, and more preferably is between 10° C. and 40° C.

Prior to the crystal precipitation or during the crystal precipitation process, an anti-solvent (e.g., diethyl ether, isopropyl ether, t-butyl methyl ether, hexane, heptane, octane or a mixed solvent thereof) may be added as appropriate.

The crystals which have precipitated in the mixed solution are filtered off, whereby the desired donepezil hydrobromide can be obtained.

The obtained crystals can be washed using the same solvent as the dissolving solvent, as required. Further, the obtained crystals can, as necessary, be dried under atmospheric pressure or reduced pressure at room temperature or under heating.

For example, donepezil hydrobromide can be obtained as crystals by: (1) charging donepezil hydrobromide with a solvent (e.g., water, an alcohol (e.g., methanol, ethanol, isopropyl alcohol or the like), an ester (e.g., methyl acetate, ethyl acetate or the like), a ketone (e.g., acetone or the like), a nitrile (e.g., acetonitrile or the like), benzene, toluene, a cyclic ether (e.g., dioxane, tetrahydrofuran or the like), N,N-dimethylformamide, dimethylsulfoxide, a halocarbon (e.g., methylene chloride or the like) or a mixed solvent thereof) and dissolving by heating at 20 to 80° C.; (2) charging the resulting mixture with diethyl ether, isopropyl ether, t-butyl methyl ether, hexane, heptane, octane or the like; and (3) cooling the resulting mixture to a temperature between −20° C. and 40° C., and then filtering off the obtained precipitate.

Donepezil hydrobromide according to the present invention, or solvate thereof, or crystal thereof, is useful in the treatment, prevention, remission or improvement of various types of senile dementia; especially Alzheimer-type senile dementia, cerebrovascular disorders associated with cerebral apoplexy (cerebral hemorrhage and cerebral infarction), cerebral arteriosclerosis, head injury, etc.; aprosexia, disturbance of speech, hypobulia, attention deficit/hyperactivity disorders, emotional disorders, memorization disorders, hallucinatory-paranoid states, behavioral changes, etc. associated with encephalitis, cerebral paralysis, and the like.

Moreover, since donepezil hydrobromide according to the present invention, or solvate thereof, or crystal thereof, has a potent and highly selective anticholinesterase action, they are useful as a pharmaceutical based on its action. In addition to Alzheimer-type senile dementia, donepezil hydrobromide according to the present invention, or solvate thereof, or crystal thereof, is especially effective for, for example, Huntington's chorea, Pick's disease, and other such late-onset disorders.

When using donepezil hydrobromide according to the present invention, or solvate thereof, or crystal thereof as a pharmaceutical, such pharmaceutical is produced by mixing the salt, solvate, or crystals thereof with a suitable additive. However, the above description should not be construed as denying the use of the salt according to the present invention, or solvate thereof, or crystal thereof, in its original form.

Examples of the above additive include additives which are commonly used in pharmaceuticals, such as fillers, binders, lubricants, disintegrators, coloring agents, flavoring agents, emulsifiers, surfactants, solubilizing agents, suspending agents, isotonizing agents, buffers, antiseptics, antioxidants, stabilizers, absorption promoters and the like. These agents can be used in a suitable combination thereof as desired.

When using the salt of donepezil and hydrogen bromide according to the present invention, or solvate thereof, or crystal thereof, as a pharmaceutical, it may be administered orally or parenterally. The dosage varies depending on the severity of symptom; age, sex, body weight, and sensitivity of a patient; administration mode; administration timing, interval, and the nature, formulation and type of a pharmaceutical preparation; the kind of an active substance and other factors. Generally, the agent may be, but is not particularly limited to, administered once or between twice and four times daily at a daily dose for a normal adult of about 0.1 to 300 mg, and preferably from 1 to 100 mg.

To produce the salt of donepezil and hydrogen bromide according to the present invention, or solvate thereof, or crystal thereof, the dosage form may be produced by an ordinary method in the art of pharmaceutical preparation as an injection, suppository, sublingual tablet, tablet, capsule, coated tablet or the like. In the case of preparing as an injection, pH adjusting agents, buffers, suspending agents, solubilizing agents, stabilizers, isotonizing agents, preservatives and the like may be added to the principal ingredient as necessary, and the injection may be formed as an intravenous, subcutaneous or intramuscular injection in accordance with usual procedure. At such time, if necessary the injection can be formed as a freeze-dried product.

Examples of the suspending agent include, for example, methylcellulose, polysorbate 80, hydroxyethylcellulose, gum arabic, tragacanth powder, sodium carboxymethylcellulose, polyoxyethylenesorbitan monolaurate and the like.

Examples of the solubilizing agent include, for example, polyoxyethylene hydrogenated castor oils, polysorbate 80, nicotinamide, polyoxyethylenesorbitan monolaurate, magrogol, castor oil fatty acid ethyl esters and the like.

Examples of the stabilizer include, for example, sodium sulfite, sodium metasulphite, ethers and the like. Examples of the preservative include, for example, methyl paraoxybenzoate, ethyl paraoxybenzoate, sorbic acid, phenol, cresol, chlorocresol and the like.

The salt of donepezil and hydrogen bromide according to the present invention, or solvate thereof, or crystal thereof, can, for example, be produced by the methods disclosed in the below Examples. The effects of such compounds can be confirmed using the methods described in the Document (JP-A-64-79151) or the like. However, these are merely meant to be illustrative, and the present invention is by no means meant to be restricted to the below specific examples. The present invention may be altered as long as such alterations do not depart from the scope of the present invention.

The term “room temperature” in the below Reference Examples and Examples usually indicates from about 10° C. to about 35° C. Unless otherwise stated, the “%” represents weight percent.

### EXAMPLE 1

240 mL of ethanol was added to 30 g of donepezil, and the resulting mixture was dissolved under heating (external temperature of 50° C.). Next, while stirring at an internal temperature of 34° C., to the mixture, a mixed solution consisting of 16.5 g of hydrobromic acid (47% aqueous solution) and 60 mL of ethanol was added. This resulting mixture was further stirred for 12 hours and 20 minutes at room temperature. The resulting precipitate was filtered off, and washed with 70 mL of ethanol. The washed precipitate was then dried under reduced pressure for 7 hours at 50° C., whereby 35.2 g of donepezil hydrobromide crystals (96.7% yield) was obtained.

### EXAMPLE 2

15 mL of a water-methanol mixed solution (50:50) was added to 1.5 g of donepezil hydrobromide, and the resulting mixture was dissolved under heating (external temperature of 70° C.). Next, the resulting mixture was left for about 5 minutes at an external temperature of 5° C., and then further left overnight at an external temperature of 3° C. The resulting precipitate was filtered off and washed with 1 mL of cool water. The washed precipitate was then dried under reduced pressure for 6 hours at room temperature, whereby 0.883 g of donepezil hydrobromide salt crystals (58.9% yield) was obtained.

### EXAMPLE 3

50 mL of a water-methanol mixed solution (50:50) was added to 1.8 g of donepezil hydrobromide, and the resulting mixture was dissolved by applying ultrasonic waves under heating (external temperature of 70° C.). Approximately 4 mL of the resulting mixture was dried in a vacuum, and to this dried mixture, 20 mL of ethanol was added to dissolve the mixture. Next, the resulting mixture was left overnight at an external temperature of 3° C. The resulting precipitate was filtered off and washed with 1 mL of cool water. The washed precipitate was then dried overnight at 60° C., whereby donepezil hydrobromide crystals were obtained.

### Measurement of Powder X-Ray Diffraction Pattern

Powder X-ray diffraction pattern measurement of the crystals obtained in each of Examples was carried out in accordance with the powder X-ray diffraction measurement method described in the general test methods of the Japanese Pharmacopoeia, under the following measurement conditions.

### (Device)

Rigaku X-ray DTA System: RINT-2000 (manufactured by Rigaku Corporation)

### (Operational Method)

The samples were measured under the following conditions.

X-ray used: CuKα ray
Tube voltage: 40 kV
Tube current: 200 mA
Divergent slit: ½ deg
Light receiving slit: 0.3 mm
Scattering slit: ½ deg
Scan speed: 2°/min
Scanning step: 0.02°
Measuring range (2θ): 5 to 40°

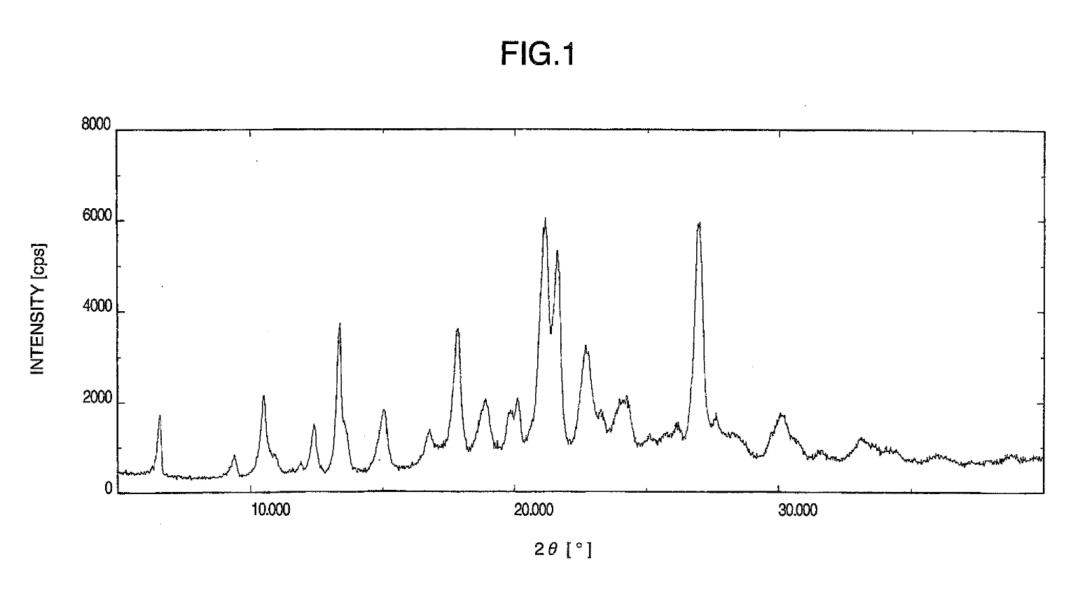

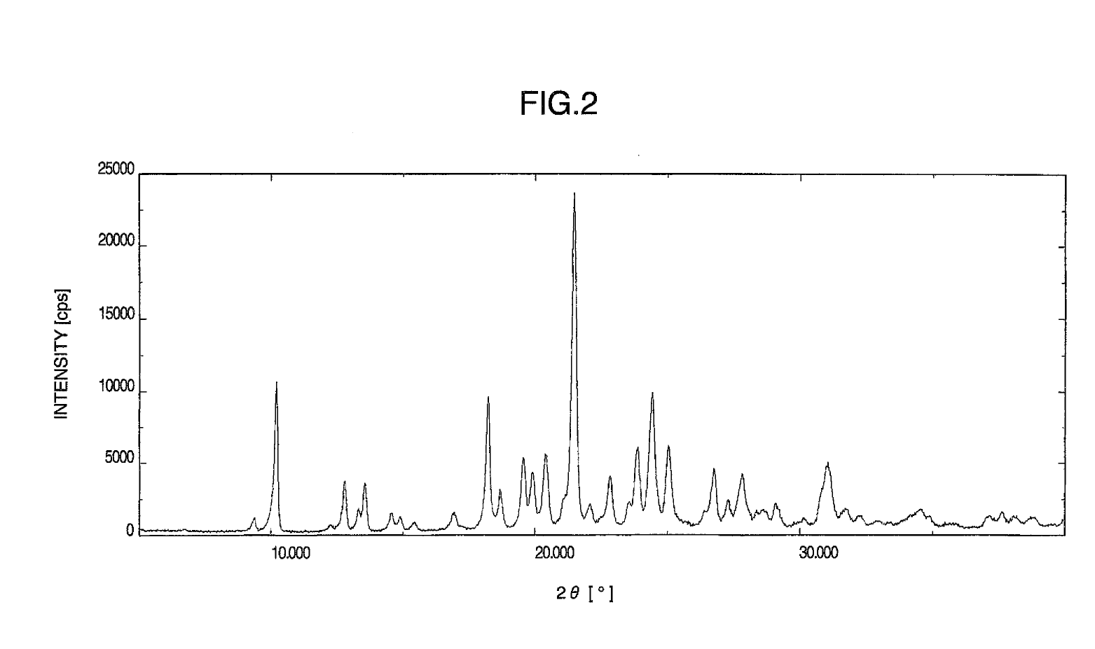

The powder X-ray diffraction pattern of the crystals obtained in Example 2 is shown in FIG. 1, and the powder X-ray diffraction pattern of the crystals obtained in Example 3 is shown in FIG. 2.

The peak and intensity of the diffraction angle (2θ) of the crystals obtained in Example 2 are shown in Table 1, and the peak and intensity of the diffraction angle (2θ) of the crystals obtained in Example 3 are shown in Table 2.

### INDUSTRIAL APPLICABILITY

Since the salt of donepezil and hydrogen bromide according to the present invention, or a solvate thereof, or a crystal thereof, has an excellent acetylcholinesterase inhibitory action, the salt is useful as a pharmaceutical, and especially useful as a preventative drug or therapeutic drug for various types of senile dementia due to its acetylcholinesterase inhibitory action.

1. 1-Benzyl-4-[(5,6-dimethoxy-1-indanon)-2-yl]methylpiperidine hydrobromide or a solvate thereof.

2. A crystal of 1-benzyl-4-[(5,6-dimethoxy-1-indanon)-2-yl]methylpiperidine hydrobromide or a solvate thereof having diffraction peaks at diffraction angles (2θ±0.2°) of 6.6° and 26.9° in powder X-ray diffraction.

3. The crystal according to claim 2, further having diffraction peaks at diffraction angles (2θ±0.2°) of 21.1° and 21.6° in powder X-ray diffraction.

4. A crystal of 1-benzyl-4-[(5,6-dimethoxy-1-indanon)-2-yl]methylpiperidine hydrobromide or a solvate thereof having diffraction peaks at diffraction angles (2θ±0.2°) of 21.5° and 24.4° in powder X-ray diffraction.

5. The crystal according to claim 4 further having diffraction peaks at diffraction angles (2θ±0.2°) of 10.2° and 18.2° in powder X-ray diffraction.

6. A method for producing the crystal according to claim 4, characterized by performing crystallization with one or two solvents selected from the group consisting of alcohol solvent and water.
